(19) United States
(12) Patent Application Publication
(10) Pub. No.: US 2023/0184109 A1
(43) Pub. Date: Jun. 15, 2023

(54) REAL-TIME MONITORING OF MICROBIAL CONTAMINATION IN OILFIELD DRILLING FLUIDS AND CEMENT

(71) Applicant: Saudi Arabian Oil Company, Dhahran (SA)

(72) Inventors: Abdulmohsen A. Al-Humam, Dammam (SA); Abdalla Moustafa Ezzat, Spring, TX (US); Abdulaziz A. Ba Hamdan, Dammam (SA)

(21) Appl. No.: 17/550,084

(22) Filed: Dec. 14, 2021

(51) Int. Cl. E21B 47/005; E21B 41/02; E21B 49/08; E21B 49/00

(52) U.S. Cl. CPC E21B 47/005; E21B 41/02; E21B 49/088; E21B 49/0875; E21B 49/005; G01N 33/2823

(57) ABSTRACT

A method for monitoring microbial levels in a wellbore fluid is provided. The method includes collecting a wellbore fluid sample, recovering ATP from the wellbore fluid sample, using the recovered ATP in an ATP-mediated oxidation of luciferin to oxyluciferin to yield photons, quantifying the photons, correlating the quantified photons to an ATP concentration, comparing the ATP concentration to predetermined action levels, and taking countermeasures when the ATP concentration exceeds the predetermined action levels.

### TECHNICAL FIELD

This document relates to methods and compositions used monitor microbial levels in oilfield drilling fluids and cement.

### BACKGROUND

Microorganisms present in oil and natural gas fields can cause a number of problems, for example, microbiologically induced corrosion or flash setting of cement. These problems can lead to significant costs for the oil and natural gas industries. Monitoring, quantifying, and treating microbes in subterranean formations and drilling fluids is an important endeavor.

### SUMMARY

This disclosure describes a method for rapidly quantifying bacteria or microbes in a drilling fluid, environmental, corrosion product, and/or drilling sample, in real-time and on-site. The quantified bacteria can then be mitigated in a timely manner.

In some implementations, a method for monitoring microbial levels in a wellbore fluid includes collecting a wellbore fluid sample, recovering ATP from the wellbore fluid sample, using the recovered ATP in an ATP-mediated oxidation of luciferin to oxyluciferin to yield photons, quantifying the photons, correlating the quantified photons to an ATP concentration, comparing the ATP concentration to predetermined action levels, and taking countermeasures when the ATP concentration exceeds the predetermined action levels.

This aspect, taken alone or combinable with any other aspect, can include the following features. The wellbore fluid is a make-up water.

This aspect, taken alone or combinable with any other aspect, can include the following features. The wellbore fluid is a cement mix fluid.

This aspect, taken alone or combinable with any other aspect, can include the following features. The wellbore fluid is a drilling mud.

This aspect, taken alone or combinable with any other aspect, can include the following features. Recovering the ATP from the wellbore fluid includes filtering the sample.

This aspect, taken alone or combinable with any other aspect, can include the following features. Quantifying the photons includes quantifying the photons with a luminometer.

This aspect, taken alone or combinable with any other aspect, can include the following features. Correlating the quantified photons to an ATP concentration includes preparing a sample of known ATP concentration, using the sample of known ATP concentration in an ATP-mediated oxidation of luciferin to oxyluciferin to yield photons, quantifying the photons, comparing the amount of photons generated from the sample of known ATP concentration to the amount of photons generated from the recovered ATP, and extrapolating the concentration of the recovered ATP from the known concentration of ATP.

This aspect, taken alone or combinable with any other aspect, can include the following features. Recovering ATP from the wellbore fluid sample includes sterilizing the wellbore fluid sample.

This aspect, taken alone or combinable with any other aspect, can include the following features. Taking countermeasures based on the ATP concentration relative to the predetermined action levels includes adding a biocide to the wellbore fluid.

This aspect, taken alone or combinable with any other aspect, can include the following features. Taking countermeasures when the ATP concentration exceeds the predetermined action levels includes taking countermeasures when the ATP concentration exceeds 1000 pg ATP/mL.

The details of one or more implementations of the disclosure are set forth in the accompanying drawings and the description that follows. Other features, objects, and advantages of the disclosure will be apparent from the description and drawings, and from the claims.

### DESCRIPTION OF DRAWINGS

FIG. 1 shows the linear correlation of ATP concentration versus colony forming units (CFU) in make-up water.

FIG. 2 shows the linear correlation of ATP concentration vs. CFU in cement mix fluid.

FIG. 3 shows the linear correlation of ATP concentration versus CFU for drilling mud samples.

FIG. 4 shows a flowchart of an example method 400 of monitoring microbial levels in a subterranean formation.

Like reference symbols in the various drawings indicate like elements.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Provided in this disclosure, in part, are methods and compositions for detecting and quantifying microbes in a subterranean formation or drilling fluid. Also provided are methods of mitigating damage and controlling microbial populations.

Subterranean drilling, for example, deep drilling, often requires the use of circulating fluids to facilitate the drilling process and carry cuttings and rock fragments to the surface. In this practice, microbial contamination of drilling fluids has significant impact on the drilling operation and reservoir integrity due to the introduction of exogenous microorganisms into the oil and natural gas reservoirs. Extensive microbial growth in the drilling fluids may result in significant biodegradation of drilling additives, such as starch and xanthan gum, which in turn may result in a loss in the rheological properties of the mud. The metabolic products from the degradation of these organic polymers can serve as food sources and promote the growth of surface microbes introduced in the drilling fluids, as well as naturally occurring oil field microbes, for example, sulfate-reducing prokaryotes (SRP), which include sulfate-reducing bacteria (SRB), and sulfate-reducing archaea (SRA). Consequences from excessive SRP activity include microbiologically influenced corrosion (MIC) in the production and processing equipment, biomass plugging in the formation, and hydrogen sulfide (H₂S) production deep in the formation. Hydrogen sulfide production can lead to reservoir souring and reduced product quality. Microorganisms can also significantly alter or degrade hydrocarbons in oil reservoirs, resulting in an increase in oil density, sulfur content, acidity, and viscosity. Finally, elevated microbial activity in the formation close to the wellbore can result in a poor cementing job, and adversely affect the wellbore characteristics.

Standard practices to monitor microbial contamination rely on culture-dependent techniques such as the Most Probable Number or MPN test. Although these techniques are accepted by NACE International as the standard technique for estimation of bacteria numbers in oilfield systems, these methods have several limitations. For example, these tests can underestimate the total bacterial population. In addition, these tests require long incubation times, delaying the turnover time of the results to 7 to 28 days or longer. Therefore, these culture-dependent techniques cannot provide drilling operations with real-time guidance to take prompt and proper actions.

Provided in this disclosure are methods of monitoring microbial activity using an ATP-bioluminescence method. ATP-bioluminescence methods are a rapid testing technique for the quantification of living cells. These testing techniques measure the amount of adenosine-5′-triphosphate (ATP) that is found in a sample.

ATP tests can be based on the detection of light generated a chemical reaction. The enzyme luciferase catalyzes the ATP-mediated oxidation of luciferin to oxyluciferin. During this reaction, ATP is dephosphorylated to adenosine monophosphate (AMP) and pyrophosphate, and a photon of light is released. The amount of bioluminescence (light) generated is proportional to the amount of ATP present in the sample, which is in turn proportional to the biomass of living microorganisms.

ATP-bioluminescence tests can quantify the amount of microorganisms in a sample rapidly and on-site. Accordingly, any needed mitigation protocols can be implemented in real-time, for example during drilling.

### EXAMPLE 1: DETECTION OF ATP VIA ATP-BIOLUMINESCENCE AND VALIDATION OF THE METHOD

A luminometer can be used to detect the relative light units (RLU) released during an ATP-bioluminescence test. A suitable luminometer is the PhotonMaster portable luminometer that can be used for on-site microbial analysis during drilling operations.

Two ATP-bioluminescence kits were tested for their ability to measure total microbiological concentration via ATP. The Quench-Gone Organic Modified (QGO-M) kit was tested for suitability in make-up water samples and cement mix fluid samples. The Deposit and Surface Analysis (DSA) kit was tested for suitability for use in drilling mud samples.

Make-up water, drilling mud, and cement mix fluid samples were collected from several drilling rig operations. ATP-based bioluminescence methods were tested for linearity and repeatability for detecting microbial levels in each of these three types of samples.

Make-up water, drilling mud, and cement mix fluid samples were spiked with a mixed bacteria population isolated from the original corresponding sample. The spiked microbial concentration was 10³ to 10⁵ cell/mL. Serial dilutions were performed by diluting the spike samples in sterile samples. The sterile samples were prepared by autoclaving the samples at 121° C. for 15 minutes. The diluted samples were analyzed with a plate count method (PCM) and an ATP-bioluminescence assay.

### Plate Count Method (PCM)

The plate count method (PCM) was used to quantify the cultivable, i.e., viable microbes in the sample. The results were correlated to ATP-bioluminescence measurements in the same sample. Tryptic soy agar (TSA) medium was adjusted to a pH of 7-10 and poured into agar plates. After the TSA medium solidified, 0.1 mL samples of the serial diluted samples were spread on the plates. TSA with a pH of 7 was used for make-up water and cement mix fluid samples, and TSA with a pH of 10 was used for drilling mud samples. Plates were inoculated in triplicate for each sample dilution and incubated at 37° C. for 18 hours. The average number of colony forming units (CFU) was multiplied by the dilution factor to determine the number of microbes per mL of the sample (CFU/mL).

### ATP-Bioluminescence Test Method, QGO-M Test Kit

Make-up water and cement mix fluid samples were prepared for ATP quantification using a LuminUltra QGO-M test kit. For make-up water, 20 mL of sample was analyzed. For cement mix fluid, 5 mL of sample was analyzed. After mixing the samples to ensure homogeneity, the appropriate amount of sample was taken up with a 20 mL syringe and then pushed through a 2μm filter. After the full volume of the sample was filtered, the filter was washed by adding 5 mL of a wash solution (LumiClean™) to the syringe barrel and pushing the wash solution through the filter. Next, the filter is dried by removing the filter from the 20 mL syringe and attaching the filter to a 60 mL syringe. The 60 mL syringe is used to push approximately 60 mL of air through the filter.

The microbes are then extracted from the filter by reattaching the filter to a 20 mL syringe barrel. Next, 1 mL of a lysing agent (UltraLyse 7™) is added to the barrel of the syringe and pushed through the filter to collect in a 9 mL dilution tube containing a dilution buffer (UltraLute).

Next, 100 μL from the dilute tube are added to an assay tube. 100 μL of luminase was added to the sample, swirled gently five times, and immediately inserted into a luminometer for measurement.

The measured relative light units (RLU) were converted to pg ATP/mL using the following equation:

$$( \frac{R L {U}_{s ample}}{R L {U}_{stnd} \times Volum {e}_{s ample}} ) \times 10 , 000 = cATP ( \frac{pgATP}{mL} ) \qquad Eq . 1$$

where RLU_{stnd} is the RLU generated from reacting 0.1 mL of the reference solution containing 1,000 pg ATP/mL with 0.1 mL of luciferase enzyme reagent, Volumeₛₐₘₚₗₑ is the volume of sample used for the test in mL, and the factor 10,000 is derived from the 10-fold dilution of the extracted ATP multiplied by the conversion of ng to pg.

### ATP-Bioluminescence Test Method, DSA Kit

A 1 mL sample of drilling mud was added to an extraction tube containing 5 mL of a lysis solution (UltraLyse 7). The extraction tube was capped and mixed vigorously to disperse the deposit throughout the fluid. The sample was then incubated for 5 minutes.

Next, 1 mL from the extraction tube was added to a 9 mL dilution tube containing a dilution buffer (UltraLute), avoiding foam from the top and solids from the middle of the extraction tube. 100 μL of the sample from the dilution tube was added to a new assay tube. 100 μL of luminase was added to the assay tube. The assay tube was swirled gently five times and then immediately inserted into a luminometer for measurement.

### Analysis

The repeatability of the ATP-bioluminescence measurements was tested at various dilutions of the samples in triplicate. At each dilution, the mean of the ATP concentration was calculated and the relative standard deviation (RSD) was determined by the equation:

$$R S D = 100 \times \frac{S}{\vert \overline{x} \vert}$$

wherein S is the sample standard deviation and x̅ is the ATP concentration mean.

The linearity of both of the ATP-bioluminescence measurements was assessed by plotting the log₁₀ of the bacterial plate count versus the log₁₀ of the ATP concentration of each dilution of the drilling samples. A linear regression analysis was performed and the coefficient of determination (R²) was determined.

### Results—Relative Standard Deviations

Table 1 shows the results of seven dilutions, in triplicate, of the quantification of cATP in wake-up water. The assay detected as low as 2 pg/mL of ATP in the make-up water sample diluted by 2500-fold. The average RSD of ATP measurements for the seven dilutions containing 2 to 2714 pg/mL of cATP was 12.4%.

Table 2 shows the results of eight dilutions, in triplicate, of the quantification of cATP in cement mix fluid. The average RSD of ATP measurements in the range of 2 to 15006 pg/mL was 7.4%.

Table 3 shows the results of six dilutions, in triplicate, of the quantification of cATP in drilling mud. The assay detected as low as 54 pg/g of ATP in the mud sample diluted by 1250-fold. In the range of 54 to 22162 pg/g of ATP, the average RSD for ATP measurements was 8.2%.

TABLE 1
RSD of ATP-bioluminescence test at
various dilutions of make-up water

| Dilution of | | Mean cATP | | RSD |
|---|---|---|---|---|
| Make-up Water | N | (pg/mL) | SD | (%) |
| 1:1 | 3 | 2714 | 45 | 2 |
| 1:2.5 | 3 | 996 | 146 | 15 |
| 1:5 | 3 | 647 | 81 | 12 |
| 1:25 | 3 | 121 | 12 | 10 |
| 1:50 | 3 | 53 | 7 | 13 |
| 1:250 | 3 | 10 | 1.9 | 18 |
| 1:2500 | 3 | 2 | 0.5 | 17 |

TABLE 2
RSD of ATP-bioluminescence test at variate
dilutions of cement mix fluid

| Dilution of | | Mean cATP | | RSD |
|---|---|---|---|---|
| Cement Mix Fluid | N | (pg/mL) | SD | (%) |
| 1:1 | 3 | 15006 | 1519 | 10 |
| 1:5 | 3 | 3694 | 591 | 16 |
| 1:25 | 3 | 815 | 69 | 8 |
| 1:62.5 | 3 | 307 | 8 | 3 |
| 1:125 | 3 | 153 | 8 | 5 |
| 1:625 | 3 | 34 | 3 | 9 |
| 1:1250 | 3 | 16 | 0.7 | 4 |
| 1:6250 | 3 | 2 | 0.1 | 4 |

TABLE 3
RSD of ATP-bioluminescent test at
various dilutions of drilling mud

| Dilution of | | Mean total ATP | | RSD |
|---|---|---|---|---|
| Cement Mix Fluid | N | (pg/g) | SD | (%) |
| 1:1 | 3 | 22162 | 2218 | 10 |
| 1:5 | 3 | 4416 | 279 | 6 |
| 1:25 | 3 | 1085 | 20 | 2 |
| 1:50 | 3 | 510 | 18 | 4 |
| 1:250 | 3 | 206 | 1 | 1 |
| 1:1250 | 3 | 54 | 14 | 26 |

Both methods demonstrated a very high repeatability of the ATP measurements for three types of samples, with an average RSD between 7.4 and 12.4%. Make-up water and cement mix fluid both have a very low background noise from the sample, and can detect

ATP at concentrations as low as 2 pg cATP/mL. However, background noise in drilling mud samples was higher than in make-up water and cement mix fluid samples. Background noise was compensated by averaging multiple measurements when establishing the ATP based action levels, i.e., concentrations of ATP that necessitate mitigation.

Results—Correlation between ATP and CFU

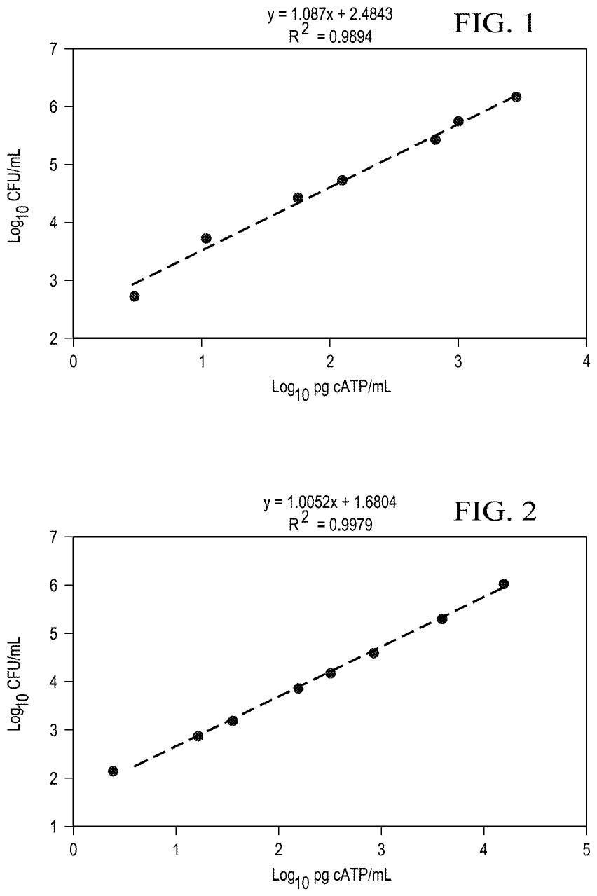

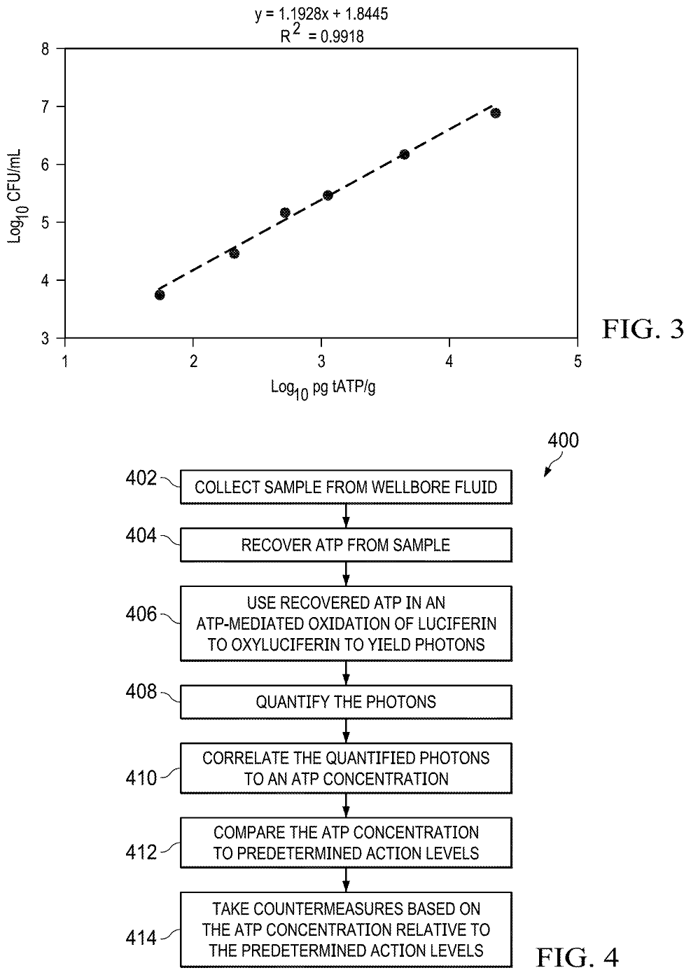

The correlation between the measured ATP (log₁₀ pg ATP/mL or pg ATP/g) and the number of cultivable microbial cells (log₁₀ CFU/mL) was determined. FIGs. AA-CC show an excellent linear correlation between the two methods, with R²>0.98 for all three types of samples. FIG. 1 shows the linear correlation of ATP concentration versus CFU in make-up water. FIG. 2 shows the linear correlation of ATP concentration vs. CFU in cement mix fluid. FIG. 3 shows the linear correlation of ATP concentration versus CFU for drilling mud samples.

### ATP-Based Action Levels

Based on microbial numbers detected from the drilling samples, and the strong correlation between the microbial numbers and the measured ATP concentration, an ATP-based action level was established for the drilling operation samples (Table 4), and the corresponding actions were summarized in Table 5. The action levels were determined to have the most protection for the tested samples. The action levels were determined by the ATP level in the tested sample and the type of tested sample. Make-up water samples have a lower action level compared to the other drilling samples, as maintaining an adequate microbial control in make-up water is critical to preventing the introduction of microbial contamination in the preparation of cement mix fluid and drilling mud.

TABLE 4
ATP-based action levels for microbial
control in drilling rig operations

| | | Preventative | Corrective |
|---|---|---|---|
| | Good Control | Action | Action |
| | (pg ATP/mL | (pg ATP/mL | (pg ATP/mL |
| Application | or/g) | or/g) | or/g) |
| Make-up water | <25 | 25 to 200 | >200 |
| Cement mix fluid | <100 | 100 to 1000 | >1000 |
| Drilling mud | <200 | 200 to 1000 | >1000 |

TABLE 5
Recommended actions for microbial
control in drilling rig operations

| Action Level | Recommendation |
|---|---|
| Good Control | The activity of microorganisms is within the specified |
| | good control limit |
| | Continue ATP monitoring |
| Preventative | The activity of microorganisms is higher than the |
| Action | specified good control limit |
| | Re-test sample |
| | Increase monitoring frequency and/or take corrective |
| | actions |
| Corrective | The activity of microorganisms is significantly higher |
| Action | than the specified good control limit |
| | Re-test sample |
| | Identify source of contamination and take corrective |
| | actions |

As described in Table 5, countermeasures include continued monitoring of the system, and, in the event of high ATP levels, biocide treatment.

FIG. 4 shows a flowchart of an example method 400 of monitoring microbial levels in a wellbore fluid. A wellbore fluid can be, for example, a make-up water, drilling fluid, fracking fluid, drilling mud, or cement mix fluid. At 402, a sample is collected from a wellbore fluid. At 404, ATP is recovered from the wellbore sample. At 406, the recovered ATP is used in the ATP-mediated oxidization of luciferin to oxyluciferin to yield photons. At 408, the photons are quantified. At 410, the quantified light is correlated to ATP concentration. At 412, the ATP concentration is compared to predetermined action levels. At 414, countermeasures are taken based on the concentration of ATP compared to the predetermined action levels.

The term “about” as used in this disclosure can allow for a degree of variability in a value or range, for example, within 10%, within 5%, or within 1% of a stated value or of a stated limit of a range.

The term “substantially” as used in this disclosure refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “solvent” as used in this disclosure refers to a liquid that can dissolve a solid, another liquid, or a gas to form a solution. Non-limiting examples of solvents are silicones, organic compounds, water, alcohols, ionic liquids, and supercritical fluids.

The term “room temperature” as used in this disclosure refers to a temperature of about 15 degrees Celsius (° C.) to about 28° C.

As used in this disclosure, the term “drilling fluid” refers to fluids, slurries, or muds used in drilling operations downhole, such as during the formation of the wellbore.

As used in this disclosure, the term “subterranean material,” or “subterranean formation” refers to any material under the surface of the earth, including under the surface of the bottom of the ocean. For example, a subterranean formation or material can be any section of a wellbore and any section of a subterranean petroleum- or water-producing formation or region in fluid contact with the wellbore. Placing a material in a subterranean formation can include contacting the material with any section of a wellbore or with any subterranean region in fluid contact the material. Subterranean materials can include any materials placed into the wellbore such as cement, drill shafts, liners, tubing, casing, or screens; placing a material in a subterranean formation can include contacting with such subterranean materials. In some examples, a subterranean formation or material can be any downhole region that can produce liquid or gaseous petroleum materials, water, or any downhole section in fluid contact with liquid or gaseous petroleum materials, or water. For example, a subterranean formation or material can be at least one of an area desired to be fractured, a fracture or an area surrounding a fracture, and a flow pathway or an area surrounding a flow pathway, in which a fracture or a flow pathway can be optionally fluidly connected to a subterranean petroleum- or water-producing region, directly or through one or more fractures or flow pathways.

As used in this disclosure, “treatment of a subterranean formation” can include any activity directed to extraction of water or petroleum materials from a subterranean petroleum- or water-producing formation or region, for example, including drilling, stimulation, hydraulic fracturing, clean-up, acidizing, completion, cementing, remedial treatment, abandonment, aquifer remediation, identifying oil rich regions via imaging techniques, and the like.

A number of implementations of the disclosure have been described. Nevertheless, it will be understood that various modifications may be made without departing from the spirit and scope of the disclosure.

What is claimed is:

1. A method for monitoring microbial levels in a wellbore fluid, comprising:
collecting a wellbore fluid sample;
recovering ATP from the wellbore fluid sample;
using the recovered ATP in an ATP-mediated oxidation of luciferin to oxyluciferin to yield photons;
quantifying the photons;
correlating the quantified photons to an ATP concentration;
comparing the ATP concentration to predetermined action levels; and
taking countermeasures when the ATP concentration exceeds the predetermined action levels.

2. The method of claim 1, wherein the wellbore fluid is a make-up water.

3. The method of claim 1, wherein the wellbore fluid is a cement mix fluid.

4. The method of claim 1, wherein the wellbore fluid is a drilling mud.

5. The method of claim 1, wherein recovering the ATP from the wellbore fluid sample comprises filtering the sample.

6. The method of claim 1, wherein quantifying the photons comprises quantifying the photons with a luminometer.

7. The method of claim 1, wherein correlating the quantified photons to an ATP concentration comprises:
preparing a sample of known ATP concentration;
using the sample of known ATP concentration in an ATP-mediated oxidation of luciferin to oxyluciferin to yield photons;
quantifying the photons;
comparing the amount of photons generated from the sample of known ATP concentration to the amount of photons generated from the recovered ATP; and
extrapolating the concentration of the recovered ATP from the known concentration of ATP.

8. The method of claim 1, wherein recovering ATP from the wellbore fluid sample comprises sterilizing the wellbore fluid sample.

9. The method of claim 1, wherein taking countermeasures based on the ATP concentration relative to the predetermined action levels comprises adding a biocide to the wellbore fluid.

10. The method of claim 1, wherein taking countermeasures when the ATP concentration exceeds the predetermined action levels comprises taking countermeasures when the ATP concentration exceeds 1000 pg ATP/mL.
